(19) United States
(12) Patent Application Publication
(10) Pub. No.: US 2015/0104505 A1
(43) Pub. Date: Apr. 16, 2015

(54) COATED PHARMACEUTICAL CAPSULE DOSAGE FORM

(71) Applicant: Cerovene, Inc., Valley Cottage, NY (US)

(72) Inventors: Manish S. SHAH, West Caldwell, NJ (US); Ray J. DiFalco, Ridgewood, NJ (US)

(21) Appl. No.: 14/502,952

(22) Filed: Sep. 30, 2014

Related U.S. Application Data
(63) Continuation of application No. 12/466,261, filed on May 14, 2009.

(51) Int. Cl. A61K 9/50; A61K 31/65

(52) U.S. Cl. CPC A61K 9/5078; A61K 9/5015; A61K 31/65
USPC 424/456; 424/463; 424/457; 514/152

(57) ABSTRACT

Pharmaceutical compositions in unit dose form comprising a hard or soft capsule containing a fill consisting of one or more inert ingredients, and one or more coatings on the capsule, wherein at least one coating comprises at least one active pharmaceutical ingredient.

### FIELD OF THE INVENTION

The present invention relates, generally, to pharmaceutical compositions in unit dose form comprising hard or soft capsules consisting of one or more inert ingredients in a pharmaceutically acceptable vehicle, and one or more coatings on the hard or soft capsule, wherein at least one coating comprises at least one active pharmaceutical ingredient, and methods of making the same.

### DESCRIPTION OF THE RELATED ART

The formulation of drugs into capsules, such as soft or hard gelatin capsules, provides a number of benefits and has been known to solve many problems associated with tableting.

In a typical conventional capsule the pharmaceutical active ingredient is present inside the capsule. The typical method of producing such conventional pharmaceutical capsule, a pharmaceutical active ingredient is mixed together with diluents such as lactose and other ingredients such as solubilizers, antioxidants, chelating agents, buffers, emulsifiers, thickening agents, dispersants, and preservatives and the mixture is then filled into hard gelatin capsules. However, some problems are known to arise with these conventional capsules. For example, hard capsules are standardized in their size and volume, and there can be technical limitations with respect to active pharmaceutical ingredients (APIs) that are to be dosed in large quantities or very small quantities. It may be difficult to achieve a homogenous mixture of drug and excipient with a uniform amount of drug present in each capsule, and a small absolute variation in the percentage of the active ingredient in the capsule can correspond to a significant variation in the dose contained in each capsule, which is clearly most undesirable. Further, manufacturing of these capsules may be expensive if more than one dosage strength of the drug needs to be made, because the drug products having multiple strengths will have different fill weights and thus require capsules of multiple different sizes. Corresponding capsule machine change parts are needed to fill the corresponding capsule size. In addition, with many drugs, there are limitations on the amount of solubilizers and surfactants that are needed to achieve the desired characteristics, such as improved bioavailability. In addition, there are sometimes problems associated with conventional capsules after administrating to patients, especially in the presence of a food, due to physiological variability relating to, for example, intrinsic properties of the active pharmaceutical ingredients.

There are several currently marketed capsule products which are filled with small spherical particles or pellets, which are coated with active pharmaceutical ingredients. One such example is Antara® Capsules, which are filled with pellets coated with fenofibrate. Other example is Oracea® Capsules, which are filled with immediate-release and delayed release pellets of doxycycline. Prilosec® Capsules are filled with delayed release pellets of omeprazole. The process of manufacturing such drug-coated pellets typically requires fluid bed technology and several coating steps to achieve the desired potency of the pellets. The coated pellets are then sieved to achieve a narrow particle size distribution. Otherwise, they produce higher weight variation during encapsulation, which is not desirable. Overall, such processes are generally relatively more expensive. The limitation with respect to the encapsulation process is same as the as the conventional capsules as mentioned earlier.

U.S. Pat. No. 7,153,538 discloses methods of coating a pharmaceutical substrate with an active coating material, where the active coating material is preferably applied electrostatically. U.S. Pat. No. 7,153,538 also discloses that conventional spray coating techniques, such as the tumble coating method, are not appropriate for use where accuracy in the amount of the active material applied to the cores is required because there is little control over the amount of coating material applied to each core.

U.S. Pat. No. 4,670,287 discloses embodiments in which a drug-filled hard capsule is selectively coated with an enteric coating agent.

U.S. Pat. No. 6,350,468 discloses a double capsule where an internal capsule is placed inside an external one, and wherein each internal and external capsule includes one or more APIs.

U.S. Pat. No. 5,641,512 discloses an analgesic soft gelatin capsule, wherein a xanthine derivative, such as caffeine, is embedded in the capsule shell itself.

U.S. Patent Application Publication No. 20070212411 discloses coated hard and soft capsules containing at least one first drug in the capsule and at least a second drug in the coating.

Japanese Patent Application Publication No. JP 59-157018 discloses capsules filled with an edible oil having a medicinal effect and coated with a powder having a medicinal effect.

All references cited herein are hereby incorporated by reference in their entirety.

### SUMMARY OF THE INVENTION

The present invention is generally directed to a pharmaceutical composition in unit dose form comprising: (a) a hard or soft capsule containing a fill consisting of one or more inert ingredients in a pharmaceutically acceptable vehicle and wherein the fill does not contain an active pharmaceutical ingredient; and (b) one or more coatings on the capsule, wherein at least one coating comprises at least one active pharmaceutical ingredient (API). The present invention is also generally directed to a pharmaceutical composition in unit dose form comprising: (a) a hard or soft capsule containing a fill consisting of one or more inert ingredients in a pharmaceutically acceptable vehicle and wherein the fill does not contain an active pharmaceutical ingredient; and (b) one or more coatings on the hard or soft capsule, wherein at least one coating comprises at least one active pharmaceutical ingredient, and wherein one or more inert ingredients in the fill increase oral bioavailability, increase solubility, delay or sustain release of one or more of the at least one active pharmaceutical ingredient.

Known capsule formulations typically have an active pharmaceutical ingredient in the capsule fill. However, unlike these known capsule formulations, embodiments of the present invention are directed to a pharmaceutical composition in which the capsule fill consists of only one or more inert ingredients. In the present invention, at least one active pharmaceutical ingredient is present in the one or more coatings on the capsule.

In some embodiments, additional coatings on the capsules, such as immediate release coatings, protective coatings, enteric or delayed release coatings, sustained release coatings, barrier coatings, and combinations thereof may be placed between the capsule and the at least one coating comprising the at least one API. In some embodiments, the capsules may be coated with at least one top coating on the at least one coating comprising the at least one API, and may include, but are not limited to, immediate release coatings, protective coatings, enteric or delayed release coatings, sustained release coatings, barrier coatings, and combinations thereof.

One or more of the APIs of the present invention may also be formulated with a combination of one or more inactive ingredients including, but not limited to, solubilizers, antioxidants, chelating agents, buffers, emulsifiers, thickening agents, dispersants, and preservatives.

### BRIEF DESCRIPTION OF THE DRAWING

FIG. 1 discloses a comparative dissolution profile of fenofibrate layered hard capsules according to Example 1 of the present invention, compared to marketed fenofibrate capsules sold under the brand name Antara®.

FIGS. 2(a) and 2(b) discloses a comparative dissolution profile of doxycycline-layered hard capsules according to Example 2 of the present invention, compared to marketed doxycycline capsules sold under the brand name Oracea®.

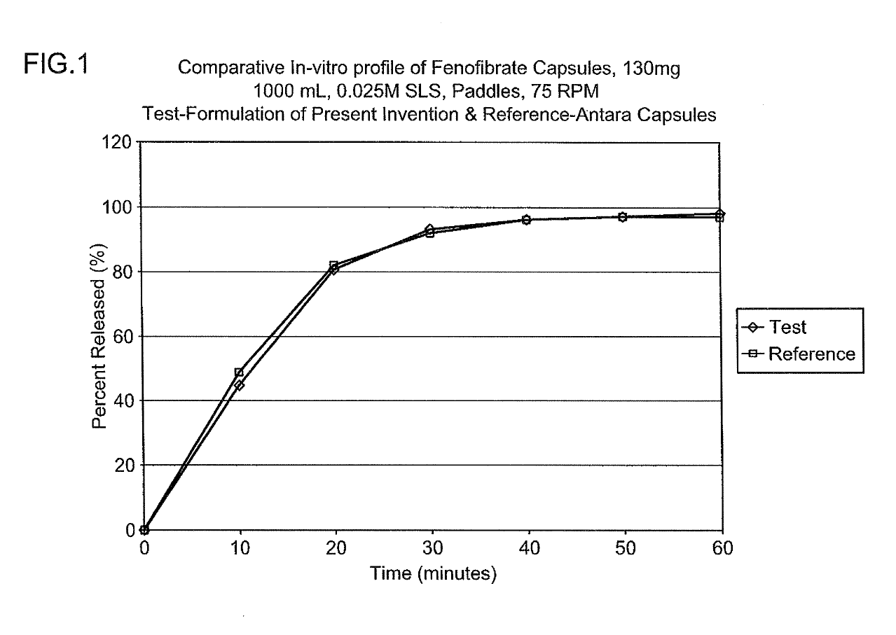

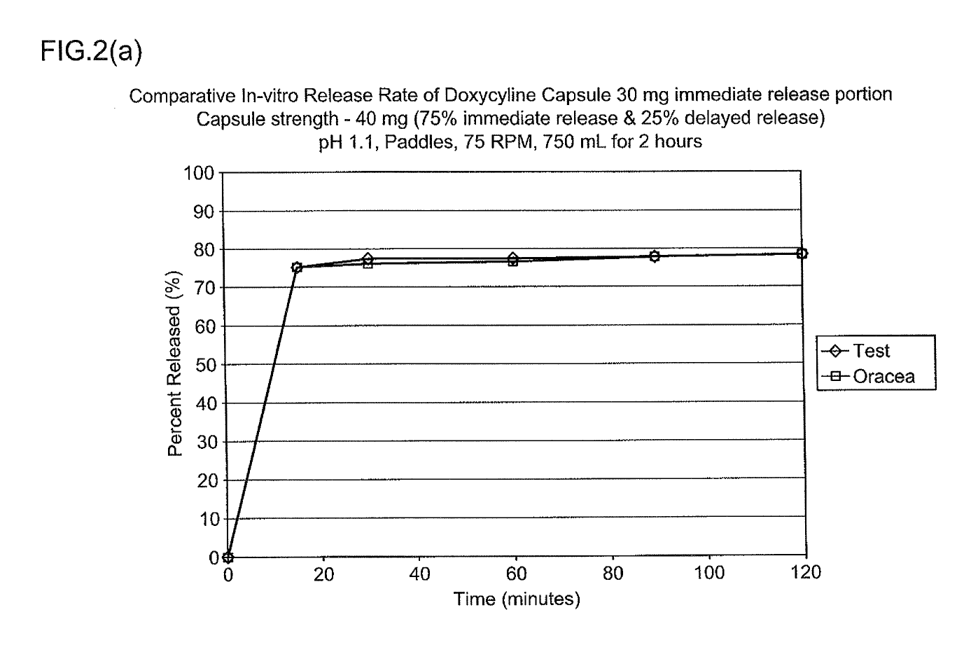

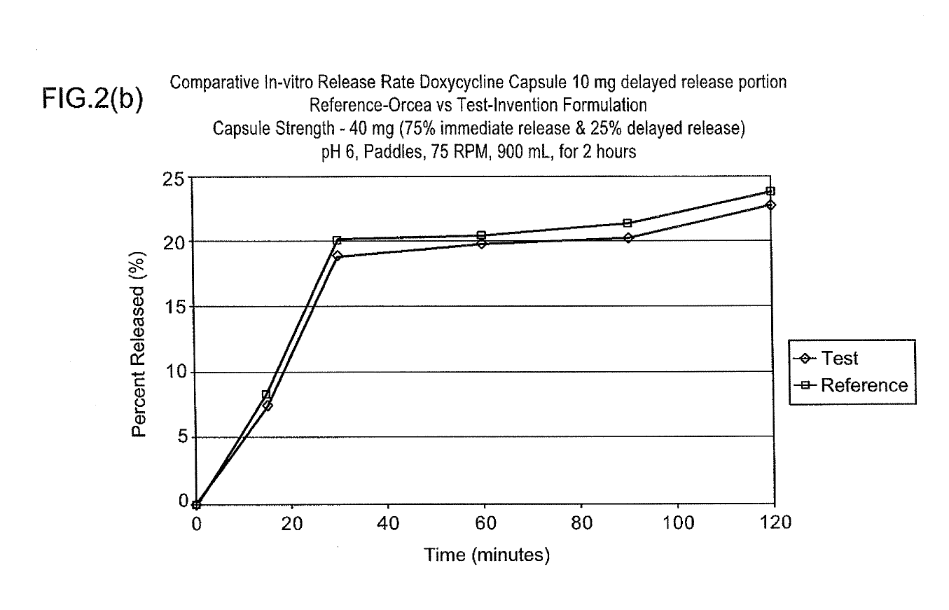

### DETAILED DESCRIPTION OF THE INVENTION

The present invention is directed to pharmaceutical compositions in unit dose form comprising: (a) a hard or soft capsule containing a fill consisting of one or more inert ingredients (also called “inactive ingredients” herein) in a pharmaceutically acceptable vehicle, and (b) one or more coatings on the capsule, wherein at least one coating comprises at least one API. The composition is suitable for oral administration.

The manufacture of hard or soft capsules is generally known by those of ordinary skill in the art. For example, soft capsules may be made by various processes including the plate process, the rotary die process, the reciprocating die process, and the continuous process. See, for example, Ebert (1978), “Soft Elastic Gelatin Capsules: A Unique Dosage Form,” Pharmaceutical Technology 1(5); Reich (2004), “Chapter 11: Formulation and physical properties of soft capsules,” Pharmaceutical Capsules, 2d Ed., Pharmaceutical Press, 201-212, hereby incorporated by reference in their entireties. See also, U.S. Pat. No. 5,478,508 and U.S. Pat. No. 5,882,680, incorporated by references herein in their entireties, disclosing methods of manufacturing seamless capsules. Examples of the capsular materials include, but are not limited to, natural or synthetic gelatin, pectin, casein, collagen, protein, modified starch, polyvinyl pyrrolidone, acrylic polymers, cellulose derivatives (such as, but not limited to, hydroxypropyl methylcellulose (HPMC)), and combinations thereof, optionally with one or more plasticizers and/or water. Capsular materials may also include one or more preservatives, coloring and opacifying agents, flavorings and sweeteners, sugars, gastroresistant substances, or combinations thereof.

The shape and size of the capsules can vary in accordance with the invention. The shape of the capsule may be, but is not limited to, round, oval, tubular, oblong, twist off, or a non-standard shape (e.g., a fish, tree, star, heart, or bear), preferably oblong. The size of the capsule used will vary in accordance to the volume of the fill composition intended to be contained therein.

For example, in some embodiments of the present invention, hard or soft gelatin capsules may be manufactured in accordance with conventional methods as a single body unit comprising the standard capsule shape. A single-body soft gelatin capsule typically may be provided, for example, in sizes from 3 to 22 minims (1 minimim being equal to 0.0616 ml) and in shapes of oval, oblong or others. The gelatin capsule may also be manufactured in accordance with conventional methods, for example, as a two-piece hard gelatin capsule, sealed or unsealed, typically in standard shape and various standard sizes, conventionally designated as (000), (00), (0), (1), (2), (3), (4), and (5). The largest number corresponds to the smallest size.

In the present invention, one or more coatings of the pharmaceutical composition comprise one or more active pharmaceutical ingredients, or APIs.

The term “active pharmaceutical ingredient,” or API, includes any compound or drug which has pharmacological or biological activity.

In some embodiments, APIs include, but are not limited to, the following: analgesics, anti-inflammatory agents, anti-helminthics, anti-arrhythmic agents, anti-asthma agents, anti-bacterial agents, anti-viral agents, anti-coagulants, anti-dementia agents, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, immunosuppressants, anti-protozoal agents, anti-thyroid agents, anti-tussives, anxiolytics, sedatives, hypnotics, neuroleptics, neuroprotective agents, β-blockers, cardic inotropic agents, cell adhesion inhibitors, corticosteroids, cytokine receptor activity modulators, diuretics, anti-Parkinson's agents, gastrointestinal agents, histamine H-receptor antagonists, keratolytics, lipid regulating agents, muscle relaxants, nitrates and other anti-anginal agents, non-steroid anti-asthma agents, nutritional agents, opioid analgesics, sex hormones, stimulants and anti-erectile dysfunction agents.

In some preferred embodiments, the API comprises a fibrate such as fenofibrate, an antibiotic such as doxycycline, a proton pump inhibitor such as omeprazole, a prostate drug such as dutasteride, an anti-inflammatory drug such as celecoxib and a cancer drug such as thalidomide

The term “inert ingredient” refers to any compound or compounds which are not active pharmaceutical ingredients. The term “inert ingredient” refers to any compound or compounds which, in the amount being used, alone does not have pharmacological or biological activity. For example, the term “inert ingredient” includes pharmaceutically acceptable excipients. In some embodiments of the present invention, unlike many capsule formulations known in the art, the fill in the hard or soft capsule only contains inert ingredients, and does not contain any API.

In some embodiments of the present invention, the inert ingredients in the capsule fill enhance the effects of the at least one active pharmaceutical ingredients in the one or more coatings on the capsule. In some embodiments, the inert ingredients in the capsule fill may enhance or increase the oral bioavailability of the active pharmaceutical ingredient.

In some embodiments, the inert ingredients can increase the solubility of the active pharmaceutical ingredient by at least 5%, more preferably at least 10%, and most preferably at least 25%. For example, sodium lauryl sulfate can be used in the capsule fill to increase the solubility of fenofibrate present in a coating of the capsule.

In some embodiments, the inert ingredients can sustain or delay the release of the active pharmaceutical ingredient. For example, the capsule fill may contain inert ingredients, which, upon administration and contact with fluids such as gastrointestinal fluids, can absorb the fluids, and associate with the particles of active pharmaceutical ingredient and sustain or delay release.

In some embodiments, upon administration of the pharmaceutical composition, the inert ingredients can change the pH of the area surrounding the active pharmaceutical ingredients and enhance the bioavailability of the active pharmaceutical ingredient. Inert ingredients which increase the pH of the surrounding area can be used to slow acid degradation, enhance the bioavailability, and/or increase the stability of acid-labile drugs. For example, sodium bicarbonate can be present in the capsule fill to enhance the bioavailability of omeprazole present in a coating of the capsule.

Examples of pharmaceutically acceptable excipients include, but are not limited to the following: anti-adhesives, inert fillers/diluents/binders, lipophilic agents and pigments. Other suitable pharmaceutically acceptable excipients are described in Remington: The Science and Practice of Pharmacy, Lippincott Williams and Wilkins, Baltimore, Md. (1995), incorporated herein by reference.

Fillers/diluents/binders may be incorporated such as sucrose, sorbitol, mannitol, various grades of lactose, various grades of microcrystalline cellulose, dextrins, maltodextrins, starches or modified starches, sodium phosphate, calcium phosphate, calcium carbonate, gelatin, polyvinylpyrrolidone, and sodium carboxymethylcellulose.

Disintegrants may be used such as cellulose derivatives, including microcrystalline cellulose, low-substituted hydroxypropyl cellulose, croscarmellose sodium, alginic acid, insoluble polyvinlypyrrolidone, and sodium carboxymethyl starch.

Glidants and lubricants may be incorporated such as stearic acid, metallic stearates, talc, waxes, and glycerides with high melting temperatures, colloidal silica, sodium stearyl fumarate, polyethyleneglycols, and alkyl sulphates.

Surfactants may be employed such as non-ionic (various grades of polysorbate); anionic such as docusate sodium and sodium lauryl sulfate, and cationic such as benzalkonium chloride. An example of an amphoteric surfactant is 1,2-diacyl-L-phosphatidylcholine. The preferred surfactants are TWEEN® 80, BRIJ®, and Nanoxyl-100.

Other appropriate pharmaceutically acceptable excipients may include colorants, flavoring agents, pH adjusting agents, solubilizing agents, wetting agents, solvent resistant agents and buffering agents.

One or more pharmaceutically acceptable excipients, may also be added to any or all of the one or more coatings, provided that they do not interfere with the drug and provide a desired benefit to the pharmaceutical. In preferred embodiments, the pharmaceutically acceptable excipients enhance the effect of the drug.

In preferred embodiments, the one or more inert ingredients enhance the activity of the active pharmaceutical ingredient. In preferred embodiments, one or more inert ingredients may be used in the composition to increase bioavailability, augment the effect of, increase the Cmax, decrease the Tmax, or otherwise benefically affect the activity of the active pharmaceutical ingredient. In some embodiments, pH adjusting agents (e.g., basifying agents or acidifying agents) such as sodium bicarbonate, calcium carbonate and tartaric acid can be used to adjust the pH in the body and increase absorption of pH-sensitive active pharmaceutical ingredients.

The term “pharmaceutically acceptable vehicle,” as used herein, includes any combination of dry and/or wet ingredients, including, but not limited to: a powder, a solid, a semisolid, an oil, a solution optionally comprising a solubilizer, a suspension, or any mixture thereof. In some embodiments, the hard or soft gelatin capsule may contain inert ingredients as a powder, granulate, beads or microtablets (e.g., similar system to U.S. Pat. No. 5,681,588, incorporated herein by reference in its entirety).

The one or more coatings or layers on the capsule may be applied by any conventional technique including, but not limited to, pan coating, fluid bed coating or spray coating. The coating(s) may be applied, for example, as a solution, suspension, spray, dust or powder. In preferred embodiments, the one or more coatings are applied by spray coating.

The present invention provides that at least one coating applied to the outside of the capsule comprises an API. In some embodiments the thickness of this layer is from 5-800 microns, preferably 10-600 microns, more preferably 20-400 microns, most preferably 40-200 microns. In some embodiments, this layer is expressed in terms of percentage weight gain, based on the total weight of the capsule including any layers provided on the capsule prior to the at least one coating comprising the API. This layer may have a weight gain of 0.05-80%, preferably 0.1-60%, more preferably 1-50%, and most preferably 5-20%.

Some embodiments of the present invention provide that the at least one coating comprising the API includes an amount of at least one compound sufficient to improve the solubility of the at least one active pharmaceutical ingredient for a pharmaceutically acceptable duration of time. In some embodiments, the at least one compound comprises at least one polymer. The amount of polymer(s) to the amount of the API is preferably from about 1:20 to about 20:1 by weight, preferably from 1:5 to about 10:1 by weight. In embodiments where the amount of API is less than about 15 mg, the amount of polymer(s) is preferably from about 1:2 to about 5:1, and more preferably from about 1:1 to about 4:1. In embodiments where the amount of API is about 20 mg or more, the amount of polymer(s) is preferably about 1:4 to about 4:1, and more preferably about 1:3 to about 2:1. The polymers may include any pharmaceutically acceptable polymers known to those of skill in the art. Preferred polymers include, but are not limited to, cellulose derivatives such as hydroxyethyl cellulose, hydroxypropyl cellulose, hydroxypropyl methylcellulose, polyvinylpyrrolidone, polyvinylpyrrolidone/vinyl acetate copolymer, ethyl cellulose aqueous dispersions and combinations thereof, preferably hydroxpropyl cellulose, ethyl cellulose, and mixtures thereof. The preferred polymers may also include one or more of the polymers disclosed throughout the application or mixtures thereof.

In some embodiments of the present invention, the API is provided in a coating solution or suspension which is applied to the capsule. In preferred embodiments, the API is provided in a homogenous coating solution or a heterologous suspension in a pharmaceutically acceptable solvent, preferably an aqueous or organic solvent. Pharmaceutically acceptable organic solvents have the advantages that they may be evaporated or sublimated during production, do not deform, melt, or otherwise change the structure of the capsule (e.g., gelatin in a soft gelatin capsule), and do not generally cause agglomeration of the coated capsules. In preferred embodiments, the pharmaceutically acceptable organic solvent is selected from methanol, ethanol, isopropranol, ethylene glycol, acetone, or mixtures thereof.

Additional pharmaceutically acceptable organic solvents that may be used include, but are not limited to, polypropylene glycol; polypropylene glycol; polyethylene glycol (for example, polyethylene glycol 600, polyethylene glycol 900, polyethylene glycol 540, polyethylene glycol 1450, polyethylene glycol 6000, polyethylene glycol 8000 (all available from Union Carbide), and the like); pharmaceutically acceptable alcohols which are liquids at about room temperature (for example, propylene glycol, ethanol, 2-(2-ethoxyethoxyl)ethanol (TRANSCUTOL™, Gattefosse, Westwood, N.J. 07675), benzyl alcohol, glycerol, polyethylene glycol 200, polyethylene glycol 300, polyethylene glycol 400 and the like); polyoxyethylene castor oil derivatives (for example, polyoxyethyleneglycerol triricinoleate or polyoxyl 35 castor oil (CREMOPHOR™ EL, BASF Corp.), polyoxyethyleneglycerol oxystearate (CREMOPHOR™ RH 40 (polyethyleneglycol 40 hydrogenated castor oil) or CREMOPHOR™ RH 60 (polyethyleneglycol 60 hydrogenated castor oil), BASF Corp.), and the like); saturated polyglycolized glycerides (for example, GELUCIRE™ 35/10, GELUCIRE™ 44/14, GELUCIRE™ 46/07, GELUCIRE™ 50/13 or GELUCIRE™ 53/10 and the like, available from Gattefosse, Westwood, N.J.); polyoxyethylene alkyl ethers (for example, cetomacrogol 1000 and the like); polyoxyethylene stearates (for example, PEG-6 stearate, PEG-8 stearate, polyoxyl 40 stearate NF, polyoxyethyl 50 stearate NF, PEG-12 stearate, PEG-20 stearate, PEG-100 stearate, PEG-12 distearate, PEG-32 distearate, PEG-150 distearate and the like); ethyl oleate, isopropyl palmitate, isopropyl myristate and the like; dimethyl isosorbide; N-methylpyrrolidinone; parafin; cholesterol; lecithin; suppository bases; pharmaceutically acceptable waxes (for example, carnauba wax, yellow wax, white wax, microcrystalline wax, emulsifying wax and the like); pharmaceutically acceptable silicon fluids; soribitan fatty acid esters (including sorbitan laurate, sorbitan oleate, sorbitan palmitate, sorbitan stearate and the like); pharmaceutically acceptable saturated fats or pharmaceutically acceptable saturated oils (for example, hydrogenated castor oil (glyceryl-tris-12-hydroxystearate), cetyl esters wax (a mixture of primarily C₁₄-C₁₈ saturated esters of C₁₄-C₁₈ saturated fatty acids having a melting range of about 43-47° C.), glyceryl monostearate; and the like.

The coatings may also include a coating material, such as a film forming material and/or binder, and optionally other conventional additives such as lubricants, surfactants, fillers and antiadherents. Preferred coating materials may include antioxidants, buffers, solubilizers, dyes, chelating agents, disintegrants, and/or absorption enhancers. Surfactants may act as both solubilizers and absorption enhancers. The coating(s) may be formulated for immediate release, delayed or enteric release, or sustained release of the API in accordance with methods well known in the art. Conventional coating techniques are described, e.g., in Remington's Pharmaceutical Sciences, 18th Ed. (1990), hereby incorporated by reference.

Additional coatings to be employed in accordance with the invention may include, but are not limited to, for example, one or more immediate release coatings, protective coatings, enteric or delayed release coatings, sustained release coatings, barrier coatings, and combinations thereof.

An immediate release coating is coating which can rapidly release the drug from the dosage form. Rapid breakdown of the film in gastric media is important, leading to effective disintegration and dissolution. Eudragit RD100 (Rohm) is an example of such a coating. It is a combination of a water insoluble cationic methacrylate copolymer and a water soluble cellulose ether. In powder form, it is readily dispensable into an easily sprayable suspension that dries to leave a smooth film. Such films rapidly disintegrate in aqueous media at a rate that is independent of pH and film thickness.

A protective coating layer (i.e., seal coat) may be applied, if desired, by conventional coating techniques such as pan coating or fluid bed coating using solutions of polymers in water or suitable organic solvents or by using aqueous polymer dispersions. Suitable materials for the protective layer include cellulose derivatives such as hydroxyethyl cellulose, hydroxypropyl cellulose, hydroxypropyl methylcellulose, polyvinylpyrrolidone, polyvinylpyrrolidone/vinyl acetate copolymer, ethyl cellulose aqueous dispersions and the like. The protective coating layer may include antioxidants, chelating agents, colors or dyes. One of the functions of the protective coating is that it can stabilize the drug when it is exposed to accelerated conditions of temperature and humidity. The protective coating may also provide alcohol resistance to the dosage form and thus help to prevent dose dumping of the drug.

A delayed release or enteric coating layer may be applied onto the capsule itself, or onto other coatings on the capsule, with or without seal coating, by conventional coating techniques, such as pan coating or fluid bed coating using solutions of polymers in water or suitable organic solvents or by using aqueous polymer dispersions. All commercially available pH-sensitive polymers are included. Typically in such uses, the API is not released in the acidic stomach environment of approximately below pH 4.5, but not limited to this value. The pharmaceutical active should become available when the pH-sensitive layer dissolves at the greater pH; after a certain delayed time; or after the unit passes through the stomach. If utilized, the preferred delay time is in the range of two to six hours.

Delayed release or enteric polymers include cellulose acetate phthalate, Cellulose acetate trimellitate, hydroxypropyl methylcellulose phthalate, polyvinyl acetate phthalate, carboxymethylethylcellulose, co-polymerized methacrylic acid/methacrylic acid methyl esters such as, for instance, materials known under the trade name EUDRAGIT L12.5, L100, or EUDRAGIT S12.5, S100 or similar compounds used to obtain enteric coatings. Aqueous colloidal polymer dispersions or re-dispersions can be also applied, e.g. EUDRAGIT L 30D-55, EUDRAGIT L100-55, EUDRAGIT S100, EUDRAGIT preparation 4110D (Rohm Pharma); AQUATERIC, AQUACOAT CPD 30 (FMC); KOLLICOAT MAE 30D and 30DP (BASF); EASTACRYL 30D (Eastman Chemical).

A sustained release film coat may include, but is not limited to, a water insoluble material such as a wax or a wax-like substance, fatty alcohols, shellac, zein, hydrogenated vegetable oils, water insoluble celluloses, polymers of acrylic and/or methacrylic acid, and any other slowly digestible or dispersible solids known in the art. The solvent for the hydrophobic coating material may be organic or aqueous. Preferably, the hydrophobic polymer is selected from (i) a water insoluble cellulosic polymer, such as an alkylcellulose, preferably ethylcellulose; (ii) an acrylic polymer; or (iii) mixtures thereof. In other preferred embodiments of the present invention, the hydrophobic material comprising the controlled release coating is an acrylic polymer. Any acrylic polymer which is pharmaceutically acceptable can be used for the purposes of the present invention. The acrylic polymers may be cationic, anionic or non-ionic polymers and may be acrylates, methacrylates, formed of methacrylic acid or methacrylic acid esters. Examples of suitable acrylic polymers include but are not limited to acrylic acid and methacrylic acid copolymers, methacrylic acid copolymers, methyl methacrylate copolymers, ethoxyethyl methacrylates, cynaoethyl methacrylate, methyl methacrylate, copolymers, methacrylic acid copolymers, methyl methacrylate copolymers, methyl methacrylate copolymers, methyl methacrylate copolymers, methacrylic acid copolymer, aminoalkyl methacrylate copolymer, methacrylic acid copolymers, methyl methacrylate copolymers, poly(acrylic acid), poly(methacrylic acid, methacrylic acid alkylamine copolymer, poly(methyl methacrylate), poly(methacrylic acid) (anhydride), methyl methacrylate, polymethacrylate, methyl methacrylate copolymer, poly(methyl methacrylate), poly(methyl methacrylate) copolymer, polyacrylamide, aminoalkyl methacrylate copolymer, poly(methacrylic acid anhydride), and glycidyl methacrylate copolymers.

A barrier coat may be included between the capsule and an outer coat, between outer coats, or on the outermost coat. The barrier coat may be comprised of an enteric or delayed release coat (as above) or a barrier (non-functional) layer, which serves as a protective coat and/or scavenger to prevent leaching from the shell (e.g., glycerol or water) to the outer API component or vice versa. For example, in some embodiments a barrier coat may be used to prevent leaching of glycerol and/or water inside the shell into the API.

Embodiments of the invention may also include one or more coatings on the capsule comprising one or more sequestrants, such as but not limited to, citric acid, citric acid monohydrate, dibasic sodium phosphate, phosphoric acid, potassium citrate, sodium citrate dihydrate, and the like, and/or one or more scavengers, such as but not limited to, salts or polymers preferably having ester and/or carboxylic acid groups, as known to those of skill in the art.

In some embodiments, the dosage form may be provided with a lag time between the administration of a first portion of API in one coating and the administration of second portion of API in another coating, e.g., by a delayed release or enteric coating provided as a barrier layer. In other embodiments, there is an immediate release of the first portion of the API, followed by a delayed or sustained release of the second (and/or further) portion of the API. In further embodiments, there is a delayed release of the first portion, followed by a bolus of the second (and/or further) portion.

Some preferred embodiments have at least one top coating on the coating comprising the at least one API, selected from the group consisting of immediate release coatings, protective coatings, enteric or delayed release coatings, sustained release coatings, barrier coatings, and combinations thereof.

As noted above, polymeric coatings are generally applied as aqueous-based solutions, organic-based solutions or dispersions, in which polymer-containing droplets are atomized with air or an inert gas and sprayed onto the substrate. Heated air or an inert gas may be added to the coating equipment to facilitate evaporation of the solvent and film formation. In the case of soft gelatin capsules, the processing parameters of spray rate and bed temperature must be controlled. Because gelatin is soluble in water, spraying an aqueous-based polymeric material at a high rate could lead to solubilization of the gelatin and capsule agglomeration. A high bed temperature may result in the evaporation of residual water from the capsule shell, causing the capsule to become brittle. Therefore, embodiments of the present invention comprises a method of coating soft gelatin capsules in which these consequences are avoided.

In addition, the deposition of the API onto the surface of the hard or soft capsules with high degree of accuracy could be affected by several factors. The accuracy of deposition needs to be demonstrated by evaluating coating uniformity which includes the mass variance of the coated capsules and the variance of the content of the coated API.

In general, “uniformity of dosage unit” is defined as the degree of uniformity in the amount of the drug substance among dosage units (i.e., capsules). The uniformity of dosage unit can be demonstrated by, for example, the content uniformity method or the weight variation method, as appropriate. For example, the content uniformity method is based upon an assay of the individual content of drug substance(s) in a number of individual dosage units to determine whether the individual content is within the limits set. See, for example, USP 30 <905> “Uniformity of Dosage Units” pages 378-382, which is incorporated by reference herein in its entirety. In embodiments of the present invention, content uniformity of an active ingredient (i.e., either or both of the first API and the API, preferably at least the API) is within about 15% or less of the intended dosage, preferably within about 10% or less of the intended dosage, and more preferably within about 6% or less of the intended dosage. Content uniformity of an active ingredient is preferably controlled within a factor of about 15% or less between capsules, more preferably within a factor of about 10% or less, and even more preferably within a factor of about 6% or less between capsules.

Embodiments of the present invention provide for a method of coating a hard or soft capsule containing a fill consisting of one or more inert ingredients, with at least one coating comprising an API, the method comprising controlling the rate of coating deposition on the hard or soft capsule and controlling the temperature during the coating process to produce a physically and chemically stable coated capsule. This method also allows for a content uniformity of the API within a factor of about 15% or less of the intended dose, preferably about 6% or less of the intended dose. The coating(s) of embodiments of the present invention may also be applied onto a tablet or other conventional pharmaceutical substrate.

Other embodiments of the present invention provide for a method of administering a hard or soft capsule in accordance with the invention to a subject for treatment of any of the diseases or conditions for which the API(s) may be used. For example, when the API comprises a lipid regulation agent, the method of administration may include treatment of at least one condition or disease independently selected from the group consisting of hypertriglyceridemia, hypercholesterolemia, mixed dyslipidemia, coronary heart disease (CHD), vascular disease, atherosclerotic disease and related conditions. The method of administration can also include treatment of other conditions or diseases such as, but not limited to, infections, gastrointestinal conditions, genitourinary conditions, pain or inflammation-related conditions, and cancer.

### Example 1

### Composition of a Capsule Dosage Form as Per the Present Invention

| | | |
|---|---|---|
| Item # | Ingredients | Mg/cap |

Inactive capsule formula

| | | |
|---|---|---|
| 1 | Pregelatinized Starch, NF (Starch 1500) | 213.50 |
| 2 | Hydroxypropyl methylcellulose, USP (Methocel | 60.20 |
| | E6LVP) | |
| 3 | Sodium lauryl sulfate, NF | 24.00 |
| 4 | Magnesium Stearate, NF | 3.00 |
| 5 | Empty HPMC Capsules Size # 2 | 60.00 |

Drug layering formula

| | | |
|---|---|---|
| 6 | Purified water, USP | — |
| 7 | Hydroxypropyl methylcellulose, USP (Methocel | 28.60 |
| | E6 LVP) | |
| 8 | Simethicone Emulsion Solids, USP (30% w/w | 2.08 |
| | Emulsion) | |
| 9 | Sodium lauryl sulfate, NF | 14.20 |
| 10 | Fenofibrate, USP Micronized | 130.00 |
| | Theoretical Capsule Weight | 535.58 |

The process of manufacturing the dosage form in accordance with the invention as follows:
(a) Sift Item #s 1, 2, & 3 through #40 mesh screen using a sifter.
(b) Load the sifted inactive ingredients from the previous step into a blender and blend the powders for 10 minutes.
(c) Mixing the previous step blend with the sifted (through #40 mesh) Item #4 to form a Final blend.
(d) Encapsulate the final blend into size #2 hard capsules.
(e) The drug suspension is prepared by first mixing Item #7 into item #6 until all of Item #7 is dissolved. The approximate mixing time is 60 minutes.
(f) Add Item #8 & 9 while mixing and continue for mixing for not less than 15 minutes.
(g) Add Item #10 while mixing and continue mixing for 30 minutes.
(h) Homogenize the previous step for 15 minutes using a suitable homogenizer at a medium speed.
(i) Continue mixing for not less than 15 minutes before starting the layering process.
(j) Load the inactive hard capsules from Step (d) into the coating pan.
(k) Begin layering the inactive capsules with a drug suspension prepared in
   Step (i) using the following coating pan parameters:
   Inlet temperature: 50-60 C Coating pan size: 15″
   Inlet air volume: 75 cfm Coating pan speed: 19 RPM
      Baffle: 2
(l) The color suspension may be applied on the surface of the drug layer for ease of ink printing and to avoid the direct contact with the drug during the handling of the drug product.

An optional seal layering solution consisting of hydroxypropyl methylcellulose in water can be applied on the inactive capsules before spraying the drug layering suspension. The seal layering amount of 2-5% is preferred based on the starting weight of the inactive capsules.

An optional color layering suspension can be applied on as an outer layer to avoid the exposure of the drug while handling the drug product. The preferred coat amount is the range of 2-5%.

When tested against the product marketed under the brand name Antara® capsules, the above formulation's in vitro dissolution matches very well. This demonstrates that the capsules made using a composition and process of the claimed invention are comparable but less expensive to manufacture, both in terms of time it takes to complete the batch and capital cost.

### Example 2

### Composition of a Capsule Dosage Form as Per the Present Invention

| | | |
|---|---|---|
| Item # | Ingredients | Mg/cap |

Inactive capsule formula

| | | |
|---|---|---|
| 1 | Pregelatinized Starch, NF (Starch 1500) | 54.00 |
| 2 | Lactose monohydrate (Fast-flo) | 54.00 |
| 3 | Microcrystlline cellulose (Avicel PH 102) | 70.00 |
| 4 | Magnesium Stearate, NF | 1.80 |
| 5 | Empty HPMC Capsules Size # 2 | 63.00 |

Drug layering formula

| | | |
|---|---|---|
| 6 | Purified water, USP | — |
| 7 | Hydroxypropyl methylcellulose, USP (Methocel | 3.25 |
| | E6 LVP) | |
| 8 | Simethicone Emulsion Solids, USP (30% w/w | 0.15 |
| | Emulsion) | |
| 9 | Doxycycline monohydrate, micronized | 10.00 |

Delayed-release coat formula

| | | |
|---|---|---|
| 10 | Eudragit L30D Solids (30% w/w dispersion) | 21.3 |
| 11 | Triethyl citrate | 4.26 |
| 12 | Purified Water | — |

Drug layering formula

| | | |
|---|---|---|
| 13 | Purified water, USP | — |
| 14 | Hydroxypropyl methylcellulose, USP (Methocel | 9.75 |
| | E6 LVP) | |
| 15 | Simethicone Emulsion Solids, USP (30% w/w | 0.45 |
| | Emulsion) | |
| 16 | Doxycycline monohydrate, micronized | 30.00 |

Seal-coating formula

| | | |
|---|---|---|
| 17 | Purified water, USP | — |
| 18 | Hydroxypropyl methylcellulose, USP (Methocel | 6.4 |
| | E6 LVP) | |
| | Theoretical Capsule Weight | 328.4 |

The above formulation's in-vitro dissolution when tested against the product marketed under the brand name Oracea® capsules matches very well. This demonstrates that the capsules made using a composition and process of the claimed invention are comparable but less expensive to manufacture, both in terms of time it takes to complete the batch and capital cost.

### Example 3

### Composition of a Capsule Dosage Form as Per the Present Invention

| | | |
|---|---|---|
| Item # | Ingredients | Mg/cap |

Inactive capsule Formula

| | | |
|---|---|---|
| 1 | Sodium Bicarbonate granules | 1000.00 |
| 2 | Magnesium Stearate, NF | 6.00 |
| 5 | Empty HPMC Capsules Size # 00 | 120.00 |

Drug layering formula

| | | |
|---|---|---|
| 6 | Purified water, USP | — |
| 7 | Hydroxypropyl methylcellulose, USP (Methocel | 10.00 |
| | E6 LVP) | |
| 8 | Simethicone Emulsion Solids, USP (30% w/w | 2.00 |
| | Emulsion) | |
| 9 | Omeprazole | 40.0 |

Buffered Seal-Coating formula

| | | |
|---|---|---|
| 10 | Purified water, USP | — |
| 11 | Hydroxypropyl methylcellulose, USP (Methocel | 6.0 |
| | E6 LVP) | |
| 12 | Calcium Carbonate powder | 300.0 |
| | Theoretical Capsule Weight | 1484.00 |

Omeprazole is known to be an acid liable drug which rapidly degrades in an acidic environment. Therefore, acid neutralizing agents such as calcium carbonate and sodium carbonate prevent the premature degradation of omeprazole. It is typically difficult, if not impossible, to manufacture a capsule dosage form with a high dose of sodium and calcium carbonate. The present invention allows for the manufacture of a capsule comprising a high dose of buffering agent and a drug, such as omeprazole. A currently marketed product is a powder for oral suspension available under brand name Zegerid®. This type of dosage form is not convenient.

### Example 4

### Composition of a Capsule Dosage Form as Per the Present Invention

| | | |
|---|---|---|
| Item # | Ingredients | Mg/cap |

Inactive capsule formula

| | | |
|---|---|---|
| 1 | Pregelatinized Starch, NF (Starch 1500) | 54.00 |
| 2 | Lactose monohydrate (Fast-flo) | 54.00 |
| 3 | Microcrystlline cellulose (Avicel PH 102) | 70.00 |
| 4 | Magnesium Stearate, NF | 1.80 |
| 5 | Empty HPMC Capsules Size # 2 | 63.00 |

Drug layering formula

| | | |
|---|---|---|
| 6 | Purified water, USP | — |
| 7 | Hydroxypropyl methylcellulose, USP (Methocel | 1.5 |
| | E6 LVP) | |
| 8 | Simethicone Emulsion Solids, USP (30% w/w | 0.20 |
| | Emulsion) | |
| 9 | Dutasteride powder | 0.5 |

Seal-coating formula

| | | |
|---|---|---|
| 10 | Purified water, USP | — |
| 11 | Hydroxypropyl methylcellulose, USP (Methocel | 6.4 |
| | E6 LVP) | |
| | Theoretical Capsule Weight | 251.40 |

The formulation of the present invention is capable of producing uniform, low dose drug products which meet the criteria for content uniformity. A currently marketed product sold under brand name Avodart® are soft gelatin capsules, which are far more expensive to produce.

### Example 5

### Composition of a Capsule Dosage Form as Per the Invention

| | | |
|---|---|---|
| Item # | Ingredients | Mg/cap |

Inactive capsule formula

| | | |
|---|---|---|
| 1 | Pregelatinized Starch, NF (Starch 1500) | 54.00 |
| 2 | Lactose monohydrate (Fast-flo) | 54.00 |
| 3 | Microcrystlline cellulose (Avicel PH 102) | 70.00 |
| 4 | Magnesium Stearate, NF | 1.80 |
| 5 | Empty HPMC Capsules Size # 2 | 63.00 |

Drug layering formula

| | | |
|---|---|---|
| 6 | Purified water, USP | — |
| 7 | Hydroxypropyl methylcellulose, USP (Methocel | 60.0 |
| | E6 LVP) | |
| 8 | Polyethylene glycol 400 | 10.0 |
| 9 | Simethicone Emulsion Solids, USP (30% w/w | 2.7 |
| | Emulsion) | |
| 10 | Celecoxib powder | 200.0 |

Seal-coating formula

| | | |
|---|---|---|
| 11 | Purified water, USP | — |
| 12 | Hydroxypropyl methylcellulose, USP (Methocel | 10.00 |
| | E6 LVP) | |
| | Theoretical Capsule Weight | 525.50 |

Nonsteroidal anti-inflammatory drugs (NSAIDs) are one of the most widely prescribed medications in the world. Highly selective Cox-2 selective NSAIDs such as celecoxib are ideal anti-inflammatory drugs, but they have very poor aqueous solubility and wettability and can give rise to difficulties in the design of pharmaceutical formulations, leading to variable oral bioavailability. The currently marketed product, sold under brand name Celebrex®, is a conventional capsule dosage form wherein celecoxib powder is mixed with other diluents, and the powder mix is filled inside the capsule. This may be the reason for variable oral bioavalability and longer duration of onset. A formulation in accordance with the present invention is capable of increasing solubility as the drug is already wetted and then layered on to the capsules. The overall process of producing such a coated capsule is relatively inexpensive and is able to produce the desired effect.

### Example 6

### Composition of a Capsule Dosage Form as Per the Invention

| | | |
|---|---|---|
| Item # | Ingredients | Mg/cap |

Inactive capsule formula

| | | |
|---|---|---|
| 1 | Pregelatinized Starch, NF (Starch 1500) | 54.00 |
| 2 | Lactose monohydrate (Fast-flo) | 54.00 |
| 3 | Microcrystlline cellulose (Avicel PH 102) | 70.00 |
| 4 | Magnesium Stearate, NF | 1.80 |
| 5 | Empty HPMC Capsules Size # 2 | 63.00 |

Drug layering formula

| | | |
|---|---|---|
| 6 | Purified water, USP | — |
| 7 | Hydroxypropyl methylcellulose, USP (Methocel | 60.0 |
| | E6 LVP) | |
| 8 | Simethicone Emulsion Solids, USP (30% w/w | 2.7 |
| | Emulsion) | |
| 9 | Thalidomide | 200.0 |

Seal-coating formula

| | | |
|---|---|---|
| 10 | Purified water, USP | — |
| 11 | Hydroxypropyl methylcellulose, USP (Methocel | 15.0 |
| | E6 LVP) | |
| | Theoretical Capsule Weight | 520.50 |

Thalidomide is a sedative-hypnotic, and multiple myeloma medication. The drug is a potent teratoge in rabbits and primates including humans: severe birth defects may result if the drug is taken during pregnancy. Apart from its infamous tendency to induce birth defects and peripheral neuropathy, the main side effects of thalidomide include fatigue and constipation. Currently, it is available as Thalomid® Capsule in a conventional capsule dosage form where thalidomide powder is mixed with other diluent and the resulting powder mix is then filled into appropriate size capsules. The process of manufacturing such a capsule involves several steps, due to airy nature of the drug. The drug and the diluent needs to be either slugged or chilsonated to form a dense blend so that a mixture containing the drug can be uniformly filled inside the capsule. Also due to very poor solubility, high absorption variation is possible after oral administration of a dose. The formulation and process of this invention involves a clean manufacturing operation wherein the drug is suspended in water and the suspension is layered onto the capsules. Once the drug is suspended in the water, the possibility of breathing the powder or exposure to the operators is minimal. The conventional capsule manufacturing operation is very dusty and exposure potential is much greater. The oral bioavaliability is much better as with the formulation of the invention as the drug is wetted during the manufacturing process.

What is claimed:

1. A method of treating a condition in a subject in need thereof, comprising administering to the subject a pharmaceutical composition in unit dose form comprising:
(a) a hard or soft capsule containing a fill consisting of one or more ingredients in a pharmaceutically acceptable vehicle, wherein the one or more ingredients do not have pharmacologic activity, and wherein the fill does not contain an active pharmaceutical ingredient; and
(b) one or more coatings on the hard or soft capsule, wherein at least one coating comprises at least one active pharmaceutical ingredient comprising doxycycline, and
wherein the one or more ingredients in the fill are selected from the group consisting of: pH adjusting agents, solubilizing agents, and delayed release or enteric polymers.

2. The method of claim 1, wherein the condition is selected from the group consisting of: hypertriglyceridemia, hypercholesterolemia, mixed dyslipidemia, coronary heart disease, vascular disease, atherosclerotic disease, infection, gastrointestinal conditions, genitourinary conditions, pain, and inflammation-related conditions.

3. The method of claim 1, wherein the pharmaceutical composition further comprises at least one additional coating between the capsule and the at least one coating comprising the at least one active pharmaceutical ingredient.

4. The method of claim 3, wherein the at least one additional coating is selected from the group consisting of immediate release coatings, protective coatings, enteric or delayed release coatings, sustained release coatings, barrier coatings, seal coatings, and combinations thereof.

5. The method of claim 1, wherein the pharmaceutical composition further comprises at least one top coating on the at least one coating comprising the at least one active pharmaceutical ingredient.

6. The method of claim 5, wherein the at least one top coating is selected from the group consisting of immediate release coatings, protective coatings, enteric or delayed release coatings, sustained release coatings, barrier coatings, seal coatings, and combinations thereof.

7. The method of claim 1, wherein the pharmaceutical composition further comprises at least one barrier coating between the capsule and the at least one coating comprising the at least one active pharmaceutical ingredient, and at least one top coating selected from the group consisting of enteric or delayed release coatings, and protective coatings, or combinations thereof, on the at least one coating comprising the at least one active pharmaceutical ingredient.

8. The method of claim 1, wherein the dosage form is suitable for oral administration.

9. The method of claim 1, wherein the capsule is a soft gelatin capsule.

10. The method of claim 1, wherein the at least one coating comprising the at least one active pharmaceutical ingredient is applied by spray pan coating or fluid bed coating.

11. A method of treating a condition in a subject in need thereof, comprising administering to the subject a pharmaceutical composition in unit dose form comprising:
(a) a hard or soft capsule containing a fill consisting of one or more ingredients in a pharmaceutically acceptable vehicle, wherein the one or more ingredients do not have pharmacologic activity, and wherein the fill does not contain an active pharmaceutical ingredient; and
(b) one or more coatings on the hard or soft capsule, wherein at least one coating comprises at least one active pharmaceutical ingredient comprising doxycycline, wherein the one or more coatings coats the capsule and has a thickness of 5-800 microns,
wherein the at least one coating comprising the at least one active pharmaceutical ingredient is solvent-based.

12. The method of claim 11, wherein the condition is selected from the group consisting of: hypertriglyceridemia, hypercholesterolemia, mixed dyslipidemia, coronary heart disease, vascular disease, atherosclerotic disease, infection, gastrointestinal conditions, genitourinary conditions, pain, and inflammation-related conditions.

13. The method of claim 11, wherein the pharmaceutical composition further comprises at least one additional coating between the capsule and the at least one coating comprising the at least one active pharmaceutical ingredient.

14. The method of claim 13, wherein the at least one additional coating is selected from the group consisting of immediate release coatings, protective coatings, enteric or delayed release coatings, sustained release coatings, barrier coatings, seal coatings, and combinations thereof.

15. The method of claim 11, wherein the pharmaceutical composition further comprises at least one top coating on the at least one coating comprising the at least one active pharmaceutical ingredient.

16. The method of claim 15, wherein the at least one top coating is selected from the group consisting of immediate release coatings, protective coatings, enteric or delayed release coatings, sustained release coatings, barrier coatings, seal coatings, and combinations thereof.

17. The method of claim 11, wherein the pharmaceutical composition further comprises at least one barrier coating between the capsule and the at least one coating comprising the at least one active pharmaceutical ingredient, and at least one top coating selected from the group consisting of enteric or delayed release coatings, and protective coatings, or combinations thereof, on the at least one coating comprising the at least one active pharmaceutical ingredient.

18. The method of claim 11, wherein the solvent comprises an aqueous or organic solvent.

19. The method of claim 18, wherein the solvent is selected from the group consisting of methanol, ethanol, isopropanol, ethylene glycol, acetone, and mixtures thereof.

20. The method of claim 11, wherein the at least one coating comprising the at least one active pharmaceutical ingredient further comprises a binder.

21. The method of claim 20, wherein the binder comprises hydroxyethyl cellulose, hydroxypropyl cellulose, hydroxypropyl methylcellulose, polyvinylpyrrolidone, polyvinylpyrrolidone/vinyl acetate copolymer, ethyl cellulose, or combinations thereof.

22. A method of treating a condition in a subject in need thereof, comprising administering to the subject a pharmaceutical composition in unit dose form comprising:
(a) a hard or soft capsule containing a fill consisting of one or more ingredients in a pharmaceutically acceptable vehicle, wherein the one or more ingredients do not have pharmacologic activity, and wherein the fill does not contain an active pharmaceutical ingredient; and
(b) one or more coatings on the hard or soft capsule, wherein at least one coating comprises at least one active pharmaceutical ingredient comprising doxycycline,
wherein one or more ingredients in the fill are selected from the group consisting of: pH adjusting agents, solubilizing agents, and delayed release or enteric polymers, and
wherein the at least one coating comprising the at least one active pharmaceutical ingredient is solvent-based.

23. The method of claim 22, wherein the condition is selected from the group consisting of: hypertriglyceridemia, hypercholesterolemia, mixed dyslipidemia, coronary heart disease, vascular disease, atherosclerotic disease, infection, gastrointestinal conditions, genitourinary conditions, pain, and inflammation-related conditions.

24. The method of claim 22, wherein the pharmaceutical composition further comprises at least one additional coating between the capsule and the at least one coating comprising the at least one active pharmaceutical ingredient.

25. The method of claim 24, wherein the at least one additional coating is selected from the group consisting of immediate release coatings, protective coatings, enteric or delayed release coatings, sustained release coatings, barrier coatings, seal coatings, and combinations thereof.

26. The method of claim 22, wherein the pharmaceutical composition further comprises at least one top coating on the at least one coating comprising the at least one active pharmaceutical ingredient.

27. The method of claim 26, wherein the at least one top coating is selected from the group consisting of immediate release coatings, protective coatings, enteric or delayed release coatings, sustained release coatings, barrier coatings, seal coatings, and combinations thereof.

28. The method of claim 22, wherein the solvent comprises a solvent selected from the group consisting of methanol, ethanol, isopropanol, ethylene glycol, acetone, and mixtures thereof.

29. The method of claim 22, wherein the at least one coating comprising the at least one active pharmaceutical ingredient further comprises a binder.

30. The method of claim 29, wherein the binder comprises hydroxyethyl cellulose, hydroxypropyl cellulose, hydroxypropyl methylcellulose, polyvinylpyrrolidone, polyvinylpyrrolidone/vinyl acetate copolymer, ethyl cellulose, or combinations thereof.
